(19) United States
(12) Patent Application Publication
(10) Pub. No.: US 2005/0255047 A1
(43) Pub. Date: Nov. 17, 2005

(54) Smoke composition

(75) Inventor: Philippe Rocquet, Macon (FR)

Correspondence Address: OLIFF & BERRIDGE, PLC, P.O. BOX 19928, ALEXANDRIA, VA, 22320, US

(73) Assignee: L. C. B., La Salle (FR)

(21) Appl. No.: 10/529,627

(22) PCT Filed: Oct. 2, 2003
(86) PCT No.: PCT/FR03/02893

§ 371 Date: Jul. 11, 2005

(30) Foreign Application Priority Data
Oct. 2, 2002 (FR) 02/12185

(51) Int. Cl. A61L 9/02; A61K 9/72

(52) U.S. Cl. 424/40

(57) ABSTRACT

The invention relates to a smoke composition comprising between 0.05 and 5% of one or more active ingredients, for an effective dose of active ingredient(s) of between 0.5 mg/m³ and 40 mg/m³, and a smoke base. According to the invention, the aforementioned smoke base comprises at least one oxidising agent, one reducing agent and one mineral filler. The mineral filler represents at least 25% of the weight of the base while the reducing agent represents at most 16% of the weight thereof, and the weight ratio of oxidising agent to reducing agent is at least 3/1.

The invention relates to a smoke composition comprising a smoke base and one or more active substances, generally biocidal and/or phytopharmaceutical active substances.

In the present description, the expression “biocidal and/or phytopharmaceutical active substance” is intended to mean a chemically defined and non-formulated substance, used in order to combat the presence of organisms or of microorganisms considered to be pests.

In the remainder of this patent, the expression “active ingredient of a smoke” will be used to refer to the active substance or all the active substances used in the smoke. Similarly, the term “concentration of active ingredient” will refer to the sum of each of the concentrations of the active substances, present in the smoke, out of formulation.

Fumigation is a technique which, through the combination of a substantial and abrupt increase in temperature, and a subsequent generation of gas, makes it possible to very effectively and homogeneously disperse, in the air, one or more active substances in the form of a very fine, generally at least microscopic, solid and/or liquid aerosol, or else of a gas.

Experience shows that aerosol and/or gas can get into the slightest nooks and crannies, which, combined with its great fineness, allows particularly effective treatment.

A smoke composition or preparation consists of an autoreactive base, that heats rapidly and greatly, its temperature possibly reaching several hundred degrees between a few seconds and a few minutes, while at the same time releasing a considerable amount of gas, and of one or more active substances, in the form of a powder, of a liquid or of a paste, optionally impregnated on a support, which, under the combined action of the abrupt increase in temperature and the emission of gas, will be finely dispersed, in the form of a liquid and/or solid aerosol.

The fields of application of smokes are, in general, those of disinfection and treatment with insecticide in agrofoods industries, livestock rearing, seed or harvest storage and transport, greenhouses or sheds, hospitals, community premises, domestic premises, etc. Of course, the scope of the present invention is not limited to these applications, even though its description hereinafter is given with reference to a biocidal and/or phytopharmaceutical use.

The diffusion of an active ingredient with pharmaceutical action, by inhalation, in human or veterinary medicine can, for example be envisioned.

Before continuing, some notions linked to the use of the smoke should be defined, concentrating more particularly on disinfectant or insecticidal smokes.

A smoke treatment or fumigation consists in initiating the combustion of a smoke preparation containing an active ingredient, the amount of which is in relation to the volume of the room to be treated, said room being made draughtproof beforehand.

A sufficient contact time, generally of several hours, is maintained between the smoke emissions and the closed room.

The term “fumigation time” will be used to denote the time during which the smoke effectively smokes and the term “contact time” will be used to denote the total period during which the room to be treated is effectively in contact with the emissions.

The term “fumigation” defines the treatment in its entirety.

In general, the effectiveness of a disinfectant/insecticidal smoke is determined, at the end of this predetermined contact time, either by the proportion of target organisms that have been destroyed on a surface, compared with a nontreated control, or by the quantitative decrease in the amount of damage engendered by these organisms at the smoke user's premises.

The term “effective smoke” or the expression “good effectiveness of a smoke” will be used if said smoke attains a proportion of destroyed target organisms that is compatible with quantitative requirements prefixed by standards, laws, administrative procedures, or a company's or an authority's internal regulations, or by simple experience in the field, or if the level of damage caused by the target organisms after fumigation is considered to be acceptable by the user.

The effective dose of an active ingredient is the amount of active ingredient theoretically dispersed in the atmosphere of the premises to be treated, that is necessary for an effective treatment. This dose is generally expressed in mg/m³. It corresponds in fact to the amount of active ingredient initially present in the smoke, divided by the volume to be treated. At the current time, the effective doses of active ingredient for a disinfectant smoke are of the order of 30 to 160 mg/m³ and, for an insecticidal smoke, of the order of 6 to 50 mg/m³.

The effective dose of a smoke composition is expressed by the number of grams of smoke composition per cubic meter (g/m³), that provides effective treatment within the meaning defined above. By way of illustration, the values of the effective doses for insecticidal smoke compositions are of the order of 0.2 to 2 g/m³ and, for disinfectant smoke compositions, of the order of 0.3 to 1.5 g/m³.

However, of course, the effective dose of a smoke depends on the concentration and on the effectiveness of the active ingredient, on the contact time and on the use.

The term “ground residues” is intended to mean all the products that are on the ground after fumigation, i.e. smoke degradation products, but also active ingredient and constituents of the smoke.

The term “emissions” is intended to mean all the compounds emitted into the air by the smoke base, i.e. all the aerosols and gases.

Unlike the products referred to as “fumigating products” that give off highly toxic gases and are reserved for specialist teams, or even teams subject to authorization, smokes can be handled by individuals who are not technicians, and in particular the public at large, even though, in general, the treatment per se is carried out with no human presence.

Consequently, it would be necessary to have smoke compositions which, besides good effectiveness, have the following properties:
easy to use, in particular in terms of lighting, which must be done readily, but with no flame nor an explosion,
little odor, persistent or otherwise,
few ground residues,
residues and emissions that are relatively nontoxic with respect to the environment and to humans, and that are clearly identifiable,
no projection of hot products out of the container,
entirely safe production, transport, storage and handling, in terms of both the dangers of catching light or even exploding, and the toxicity of the smoke product in itself.

Currently, a compromise is generally reached with smoke bases comprising an oxidoreductive system which engenders the action of abrupt heating and the release of gas, the oxidizing agent generally being nitrate-, chlorate- or perchlorate-based, and the reducing agent being nitrogenous compound- or carbon compound-based, and excipients that are involved in the quality of the ignition, or in the combustion so that it is even and without flames, or that facilitate the handling.

However, these compositions have various drawbacks. They still have too strong an odor during and after fumigation, and they generate considerable amounts of ground residues, that are mainly organic. The residues are sometimes toxic and dirty, and have a poorly defined general composition. In addition, they often have the effect of decreasing the effectiveness of the active ingredient. The amount of dry ground residues of these smoke compositions is generally between 5 and 15% of the initial mass thereof.

Such compositions are, for example, described in the following documents.

Document JP-03-086804 describes an insecticidal or fungicidal smoke composition, the smoke base of which comprises potassium chlorate, a reducing agent such as glucose or starch, a thiourea derivative (1-30%) and, optionally, a mineral filler chosen from clay and talc, melamine as a combustion regulating agent and carboxymethylcellulose.

Document FR-A-2 096 873 discloses a smoke composition comprising ammonium nitrate in a proportion of the order of 42% by weight of the total weight of the composition, dicyandiamide in a proportion of the order of 28%, as a reducing agent, 0.5% to 20% of a mineral filler whose function is to act as a heat screen, and from 5% to 10% of a fungicidal or bactericidal active ingredient.

According to document FR-A-2 637 459, an insecticidal, bactericidal or fungicidal smoke composition is known, the smoke base of which comprises ammonium nitrate (at least 42% by weight of the final weight of the base), dicyandiamide (at least 28% by weight) as a reducing agent, silica (approximately 10%) and a chlorinated compound in liquid or solid form.

The large amount of residues generated by these compositions of the prior art constitutes a major drawback thereof.

These residues are essentially the result of the degradation, during the fumigation, of the reducing agent and of the active ingredient, which, in these compositions, often represent, by weight, around a third, or even more, of the final composition. This degradation, besides producing residues which can lead to a certain toxicity for humans or the environment, makes it necessary to increase the amount of active ingredient initially present, in order to reach the required level of effectiveness.

To solve this problem, the applicant has sought a smoke base which, while having the abovementioned properties, namely effectiveness, easy handling and limited risks of projection, of ignition and of explosion, would make it possible to decrease the proportion of the reducing agent and of the active ingredient while increasing the effectiveness of the latter.

The applicant has thus discovered a highly effective smoke base, the properties of which will be illustrated later in the description, in which the proportion of the reducing agent is greatly reduced compared with that of the above compositions, and which makes it possible to obtain an effective composition with an amount of active ingredient that is much lower than those of the known compositions.

One of the advantages of the invention is therefore to decrease the initial amount of active ingredient used for treating one m³ of air. This means that, for the same effective dose of smoke, the proportion of active ingredient in the smoke is quite sizably decreased.

Of course, it is not a question of increasing the effective dose of smoke, for example from 1 g/m³ to 2 g/m³, in order to decrease, in the same proportion, the concentration of active ingredient in said smoke, for example from 10% to 5%.

Unexpectedly, a correct fumigation reaction with a small proportion of reducing agent is obtained by adding a large proportion of mineral filler, which, normally, is chemically barely reactive.

Just as unexpectedly, the applicant has also discovered that the use of this base makes it possible to significantly decrease the proportion of active ingredient in the air and therefore in the composition, without the effectiveness of the composition obtained being affected.

Since the applicant uses, for practical reasons, effective doses of bactericidal, fungicidal and insecticidal smoke of 0.1 g/m³ to 3 g/m³, the concentrations of active ingredient in the smoke which is the subject of the present invention are of the order of 0.05% to 5%, in order to obtain a theoretical dose of active ingredient in the air of the order of 0.5 mg/m³ to 40 mg/m³.

As mentioned above, the values of the effective doses for an insecticidal active ingredient in the current smokes are of the order of 6 to 50 mg/m³ and, for a disinfectant smoke, of the order of 30 to 160 mg/m³.

For smokes used at different doses, it would of course be necessary to correct, in proportion, the concentration of active ingredient in the smoke.

Similarly, the above doses only apply for fungicidal, bactericidal and insecticidal applications.

For other applications, it is quite obvious that said doses may be different.

Thus, a first subject of the invention is a smoke composition comprising 0.05 to 5% of one or more active ingredients, for an effective dose of active ingredient(s) of 0.5 mg/m³ to 40 mg/m³, and a smoke base; said smoke base comprises at least one oxidizing agent, one reducing agent and one mineral filler, the mineral filler representing at least 25%, advantageously between 25 and 65%, by weight of said base, the reducing agent at most 16%, advantageously between 2 and 13%, by weight of said base, and the ratio by weight of said oxidizing agent to said reducing agent being at least 3/1.

Compared with the known compositions, the proportions of reducing agent and of active ingredient are greatly reduced, and the ratio of oxidizing agent to reducing agent is high, which results in compositions for which the fumigation engenders much fewer residues.

According to the invention, the mineral filler is at least partially functional and comprises at least one aerating agent, one catalyst and/or one regulating agent.

In the remainder of the text, the term “compound” will be used for a single product, and the term “agent” will be used for a compound or a mixture of compounds satisfying at least one specific function in the fumigation.

Before disclosing in greater detail the characteristics and advantages of the invention, certain terms used in the description and the claims are defined.

An aerating agent is a compound or mixture of compounds that facilitate the evacuation of the gases during the fumigation.

It therefore prevents, during the eruption of the gases which occurs during the combustion, the formation of an overpressure which can result in hot materials being ejected out of the container.

As a result of this, it also has the effect of facilitating the exit of the active ingredient.

According to the invention, an aerating agent is in the form of a powder, with a small particle size, and with a low apparent density compared with the real density, and the shape of the particles of which promotes the passage of gases.

By way of counterexample, clay, due to the lamellar structure of its particles, is not a good aerating agent.

A combustion-regulating agent is a compound or mixture of compounds whose aim is to make sure that the fumigation is as even as possible, and to prevent said fumigation becoming too vigorous or too weak. This regulation can take place by vapors being given off which evacuate some of the energy of the oxido-reduction.

In parallel, the aim of these vapors may also be to facilitate the exit of the active ingredient, by entrainment, and to protect this same active ingredient against reactive compounds capable of degrading it, such as oxidizing gases (nitrogen oxides and N₂O).

An agent that catalyzes the combustion is a compound or mixture of compounds that facilitates the triggering and the maintenance of the reaction. The catalyst allows the fumigation to be initiated with an ignition system that is reasonably powerful, in particular in terms of risks during handling, and the reaction to be self-maintaining.

A smoke composition according to the invention emits or releases an aerosol or a gas, generally intended to destroy or to control microorganisms, insects or other unwanted invaders. It consists of a set of compounds comprising at least the smoke base and the active ingredient, the latter being advantageously chosen from bactericidal, fungicidal, virucidal, yeast-destroying, insecticidal, and acaricidal agents, and also having to possess good resistance to heat and to oxidation, and to readily vaporize.

The active ingredient is introduced into the smoke composition, in solid, liquid or pasty form.

By way of illustration, the C.E.B. insecticidal test protocol No. 135a and the airborne disinfection standard NFT 72-281 may be considered.

With a base of the invention, effectiveness according to the C.E.B. insecticidal test protocol No. 135a, for an active ingredient, at the dose of 1.5 mg/m³ is unexpectedly observed, whereas, with a conventional base and this same active ingredient, the effective dose is 9 mg/m³.

Using another base of the invention, it is also unexpectedly observed that a disinfectant smoke composition can be effective within the meaning of standard NFT-72-281 in terms of fungal and bacterial destruction, with an active ingredient at a dose of 2 mg/m³ versus 100 mg/m³ previously with the same active ingredient.

A smoke base of the invention therefore makes it possible, in comparison with the known smoke bases and at comparable effectiveness, to decrease both the proportion of the reducing agent and that of the active ingredient, which are the two main sources of residues after fumigation.

Other preferential characteristics of the invention, considered alone or in combination, and making it possible to obtain the abovementioned advantages, are disclosed hereinafter.

Thus, said base may comprise, relative to the final weight of the base, from 2% to 14%, preferably 8 to 12% by weight of said aerating agent and from 0.5 to 10%, preferably 3 to 5% by weight of said catalyst, and from 12 to 60% by weight, preferably from 23 to 42% of said regulating agent.

Certain compounds can perform several functions; they may thus be both catalysts and regulating agents or aerating agents and regulating agents. In this case, the proportion in the base of such a compound can reach the proportion of catalyst or aerating agent, plus, if necessary, the proportion of regulating agent.

Clay and magnesium chloride hexahydrate, phosphates and polyphosphates are preferential catalyst and regulating compounds.

Inorganic hydroxides, carbonates and bicarbonates are aerating and regulating compounds.

Among these, aluminum hydroxide, magnesium hydroxide and iron hydroxide, calcium carbonate, magnesium carbonate, sodium carbonate and potassium carbonate, that may or may not crystallize with water, are preferential aerating and regulating compounds.

The simple aerating compounds are advantageously chosen from silica, such as precipitated silica or vaporized silica, kieselguhr, talc, silicates, iron oxide, aluminum oxide, magnesium oxide and calcium oxide, aluminum sulfate, magnesium sulfate, calcium sulfate, potassium sulfate, sodium sulfate and barium sulfate, and mixtures of these compounds.

The simple catalyst compounds are advantageously chosen from halogenated inorganic salts, including ammonium-based salts, and/or inorganic or organic copper salts and/or titanium oxide TiO₂ and mixtures thereof.

Among these, the preferential catalyst compounds are chosen from sodium chloride, sodium iodide, potassium chloride, potassium iodide, calcium chloride, calcium iodide, magnesium chloride, magnesium iodide, ammonium chloride, ammonium iodide, copper chloride, copper iodide, cupric oxide CuO, and titanium oxide TiO₂.

The simple regulating compounds are advantageously chosen from ammonium carbonates, ammonium bicarbonates and ammonium carbamates, and inorganic salts that crystallize with water molecules, other than the above compounds defined for the aerating and regulating compounds.

The reducing agent comprises one or more organic compounds to which one or more inorganic compounds may be added.

The organic compound is advantageously chosen from carbon derivatives, in particular highly carbonated compounds of simple formula, such as charcoal, carbon black, graphite or paraffin, carbohydrates, such as starch and cellulose and derivatives, polyols such as pentaerythritol, sorbitol, glycols such as glycerol, propylene glycol, and organic acids and the salts of said acids, said acids or salts containing at most 9 carbon atoms, preferably at most 7 carbon atoms.

The term “carbohydrate” is intended to mean mono- and polyhydroxyaldehydes, mono- and polyhydroxy ketones, in particular mono- and polysaccharides, their derivatives such as mono- and polyhydroxy acids, and mono- and polyhydroxyamines.

By way of example, advantageous carbohydrates are the various sugars, starch and its derivatives, and cellulose and its derivatives.

Starch constitutes an advantageous reducing agent according to the invention.

Another advantageous reducing agent according to the invention is chosen from salified or unsalified organic hydroxy acid, such as tartaric acid.

The reducing agent may also comprise at least one other organic compound, in particular chosen from urea, dicyandiamide, melamine and cyanamide, and their salts, azodicarbonamide, guanidine and its salts, biguanide and its salts, methylcarbazate and ethylcarbazate.

The proportion of these nitrogenous compounds cannot then exceed 12% by weight relative to the base.

The reducing agent may also comprise at least one inorganic reducing compound. The inorganic compounds are advantageously chosen from sulfur and sulfur oxides, which may or may not be anhydrous; preferably, anhydrous or nonanhydrous sodium thiosulfate Na₂S₂O₃. This is advantageously present in a proportion ranging from 1 to 6% by weight relative to the weight of the base.

The oxidizing agent of said base is advantageously chosen from nitrates, nitrites, chlorates and perchlorates, iodates and periodates, and peroxides, alone or as a mixture. Its preferential proportion in said base ranges from 30 to 70% by weight, and preferably from 40 to 60%. Thus, in a preferred variant, the oxidizing agent contains ammonium nitrate, present in a proportion ranging from 40 to 60% by weight relative to the weight of the base.

The oxidizing agent may comprise from 30 to 70% by weight of said base, preferably 40 to 60%, at least of two different nitrates. For example, it comprises from 4 to 16% by weight of said base, of potassium nitrate, sodium nitrate and/or calcium nitrate.

It may also comprise from 1 to 9% by weight of said base, of copper nitrate, aluminum nitrate or magnesium nitrate. It may also comprise at least one peroxide chosen from inorganic peroxyhydrated salts.

A preferred composition according to the invention comprises a base incorporating:
from 3 to 15% by weight of starch, and/or
from 6 to 12% by weight of silica, and/or
from 2 to 7% by weight of a chlorate or of a perchlorate or of an iodate or of a periodate, and/or
from 2 to 7% by weight of a nitrite.

Better still, said base comprises from 35 to 45% by weight of ammonium nitrate, from 5 to 12% by weight of sodium nitrate or potassium nitrate, from 6 to 12% by weight of starch, from 4 to 9% by weight of a hydroxycarboxylic acid or of its salt containing at most 6 carbon atoms, from 7 to 10% by weight of silica, and the rest in clay.

Such a base may also comprise one or more halogenated salts, in a proportion ranging from 1 to 7% by weight of the base.

Another subject of the invention is a smoke composition comprising at least one smoke base as defined above and an active ingredient.

The disinfectant effectiveness of a smoke composition is measured according to a technique described below, which is the subject of AFNOR standard, NFT 72-281.

This technique consists in providing, in a room where the fumigation will take place, test microbe-carriers, i.e. plates made of a well-defined material, covered with dried solution, or inoculum, containing a known number of identical microorganisms of the same strain.

Identical control microbe-carriers, not subjected to the emissions, are themselves also placed in the room.

After a predetermined contact time with the smoke emissions, the number of microorganisms that have survived in the test and control microbe-carriers is determined by conventional microbiological methods.

The effectiveness is then given by the ratio of the number of microorganisms of the control microbe-carrier to that of the test microbe-carrier.

If the bactericidal effectiveness of a composition is measured, a product is said to be bactericidal according to this standard if the number of viable bacteria has been divided by a ratio of 10⁵ with respect to the number of viable bacteria present in the control sample.

If the fungicidal effectiveness of a composition is measured, a product is said to be fungicidal according to this standard if the number of viable molds has been divided by a ratio 10⁴ with respect to the number of viable molds in the control sample.

### EXAMPLE 1

### Disinfectant Smoke Composition According to the Invention

| | |
|---|---|
| NH₄NO₃ | 40% |
| KNO₃ | 4% |
| NA₂S₂O₃ | 1% |
| 4′-Hydroxysalicylanilide | 0.5% |
| Starch | 7% |
| Urea | _{2%} |
| TiO₂ | 1% |
| Clay | qs 100% (i.e. 44.5%) |

At the dose of 1 g/m³, for a contact period of 15 hours, this smoke composition has, according to the standard NFT 72281, an effectiveness of 1.8 log on Aspergillus niger.

### EXAMPLE 2

### Conventional Smoke Composition

| | |
|---|---|
| NH₄NO₃ | 48% |
| Dicyandiamide | 32% |
| Precipitated silica | 10% |
| 4′-Hydroxysalicylanilide | 10% |
| | 100% |

### EXAMPLE 3

### Comparison Between the Smoke Composition According to the Invention Described in Example 1 and the Conventional Smoke Composition Described in Example 2

The conventional smoke composition has a disinfectant effectiveness equivalent to that of the composition of example 1, but with a much higher dose of active ingredient per m³. In addition, the odor is much stronger, and the amount of ground residues is clearly greater.

By way of example, the ground residue of the formulation of example 1 is between 0.5% and 2% of the initial mass of the composition, whereas that of the formulation of example 2 is between 10 and 15%.

Whether the bactericidal effectiveness or the fungicidal effectiveness of the composition is measured, the composition of example 2 has an effectiveness of 1 g/m³ of initial powder, for a contact time of 15 hours.

### EXAMPLE 4

Insecticidal Effectiveness of a Smoke Containing Bifenthrin

| Formula | |
|---|---|
| Premix | |
| Al(OH)₃ | 10.0% |
| Bifenthrin | 1.5% |

The bifenthrin is premolten, so as to be absorbed onto the aluminum hydroxide. This premix is then added to the following base of the invention:

| | |
|---|---|
| NH₄NO₃ | 50.0% |
| Precipitated silica | 10.0% |
| Clay | 19.5% |
| Starch | 9.0% |
| | 100.0% |

This smoke is tested at the dose of 0.1 g/m³, therefore at 1.5 mg/m³ of bifenthrin, according to the C.E.B. protocol No. 135a.

The species tested are:
Blattella germanica
Plodia interpunctella
Tribolium confusum
Rhizopertha dominica
Musca domestica
Ctenocephalides felis.

Result: After exposure for 4 hours, none of the insects have survived.

A conventional usage dose for a smoke is 9 mg/m³ of bifenthrin, i.e. clearly more than the dose of 1.5 mg/m³ used in the smokes of the present invention.

In addition, the ground dry residue can be estimated at approximately 1% of the initial mass of the smoke, and consists essentially, besides the bifenthrin, of ammonium nitrate, which is not very toxic at all.

For this smoke composition, if premises of 1000 m³, 4 meters high, are taken into consideration, the ground dry residue will be of the order of 4 mg/m², if the surface of the walls and ceilings are not taken into account.

This will therefore be an extremely small amount.

1. A smoke composition comprising 0.05 to 5% of one or more active ingredients, for an effective dose of active ingredient(s) of 0.5 mg/m³ to 40 mg/m³, and a smoke base, characterized in that the smoke base comprises at least one oxidizing agent, one reducing agent and one mineral filler, the mineral filler representing at least 25% by weight of said base, the reducing agent at most 16% by weight of said base, and the ratio by weight of said oxidizing agent to said reducing agent being at least 3/1.

2. The smoke composition as claimed in claim 1, characterized in that said base comprises from 2% to 13% by weight of reducing agent, and from 25% to 65% by weight of mineral filler.

3. The smoke composition as claimed in claim 1, characterized in that the mineral filler comprises at least one aerating agent, one catalyst and one regulating agent.

4. The smoke composition as claimed in claim 3, characterized in that said base comprises, relative to the final weight of the base, from 2 to 14%, preferably 8 to 12% by weight of said aerating agent, from 0.5 to 10%, preferably 3 to 5% by weight of said catalyst, and from 12 to 60% by weight, preferably from 23 to 42% of said regulating agent.

5. The smoke composition as claimed in claim 3, characterized in that the compounds of the aerating agent are chosen from silica, kieselguhr, silicates, iron oxide, aluminum oxide, magnesium oxide and calcium oxide, talc, aluminum sulfate, magnesium sulfate, calcium sulfate, potassium sulfate, sodium sulfate and barium sulfate, and mixtures of these compounds.

6. The smoke composition as claimed in claim 5, characterized in that the silica is precipitated or vaporized silica.

7. The smoke composition as claimed in claim 3, characterized in that at least one catalyst compound of the catalyst is chosen from halogenated inorganic salts, including ammonium-based salts, inorganic or organic copper salts, and titanium oxide TiO₂.

8. The smoke composition as claimed in claim 7, characterized in that at least one catalyst compound of the catalyst is chosen from sodium chloride, sodium iodide, potassium chloride, potassium iodide, calcium chloride, calcium iodide, magnesium chloride, magnesium iodide, ammonium chloride, ammonium iodide, copper chloride, copper iodide, cupric oxide CuO, and titanium oxide TiO₂.

9. The smoke composition as claimed in claim 3, characterized in that at least one compound of the catalyst is also a compound of the regulating agent.

10. The smoke composition as claimed in claim 9, characterized in that said “compound at least” of the catalyst and of the regulating agent is chosen from clay, magnesium chloride hexahydrate, phosphates and polyphosphates, that may or may not be hydrated.

11. The smoke composition as claimed in claim 10, characterized in that the “compound at least” of the catalyst and of the regulating agent is clay, in all its forms, including kaolin.

12. The smoke composition as claimed in claim 3, characterized in that at least one compound of the aerating agent is also a compound of the regulating agent.

13. The smoke composition as claimed in claim 12, characterized in that said “compound at least” of the aerating agent and of the regulating agent is chosen from inorganic hydroxides, carbonates and bicarbonates.

14. The smoke composition as claimed in claim 13, characterized in that said “compound at least” of the aerating agent and of the regulating agent is chosen from magnesium hydroxide, aluminum hydroxide, sodium carbonate, potassium carbonate, calcium carbonate and magnesium carbonate, that may or may not crystallize with water.

15. The smoke composition as claimed in claim 3, characterized in that the regulating agent also comprises compounds chosen from ammonium carbonates, ammonium bicarbonates and ammonium carbamates, and inorganic salts that crystallize with water molecules, other than magnesium hydroxide, aluminum hydroxide, sodium carbonate, potassium carbonate, calcium carbonate and magnesium carbonate.

16. The smoke composition as claimed in claim 3, characterized in that the reducing agent comprises at least one organic compound.

17. The smoke composition as claimed in claim 16, characterized in that said “organic compound at least” is chosen from carbohydrates and derivatives, polyols, organic acids and the salts of said acids, said acids or salts containing at most 9 carbon atoms, and carbon derivatives, such as charcoal, carbon black or graphite.

18. The smoke composition as claimed in claim 17, characterized in that said “organic compound at least” is starch, sorbitol, glycerol or pentaerythritol.

19. The smoke composition as claimed in claim 17, characterized in that said “organic compound at least” is chosen from organic hydroxycarboxylic acids and their salts, said acids or salts containing up to 7 carbon atoms.

20. The smoke composition as claimed in claim 19, characterized in that the reducing agent comprises at least one other organic reducing compound that is chosen from urea, dicyandiamide, melamine and cyanamide, and salts thereof, azodicarbonamide, guanidine and its salts, biguanide and its salts, methylcarbazate and ethylcarbazate, said organic reducing compound being present in a maximum proportion of 12% by weight relative to the weight of the base.

21. The smoke composition as claimed in claim 1, characterized in that the reducing agent comprises at least one inorganic reducing compound.

22. The smoke composition as claimed in claim 21, characterized in that the inorganic reducing compound is chosen from sulfur and sulfur oxides, that may or may not be anhydrous.

23. The smoke composition as claimed in claim 22, characterized in that the inorganic reducing compound is anhydrous or nonanhydrous sodium thiosulfate Na₂S₂O₃.

24. The smoke composition as claimed in claim 23, characterized in that the sodium thiosulfate is present in a proportion ranging from 1 to 6% by weight relative to the weight of the base.

25. The smoke composition as claimed in claim 1, characterized in that the oxidizing agent is chosen from nitrates, nitrites, chlorates and perchlorates, iodates and periodates, and peroxides, alone or as a mixture.

26. The smoke composition as claimed in claim 1, characterized in that the oxidizing agent is present in said base in a proportion ranging from 30 to 70% by weight, and preferably from 40 to 60%.

27. The smoke composition as claimed in claim 25, characterized in that the oxidizing agent contains ammonium nitrate, present in a proportion ranging from 40 to 60% by weight relative to the weight of the base.

28. The smoke composition as claimed in claim 25, characterized in that the oxidizing agent comprises from 30 to 70% by weight of said base, preferably 40 to 60%, at least of two different nitrates.

29. The smoke composition as claimed in claim 28, characterized in that the oxidizing agent comprises from 4 to 16% by weight of said base, of potassium nitrate, sodium nitrate and/or calcium nitrate.

30. The smoke composition as claimed in claim 28, characterized in that the oxidizing agent also comprises from 1 to 9% by weight of said base, of copper nitrate, aluminum nitrate or magnesium nitrate.

31. The smoke composition as claimed in claim 25, characterized in that the oxidizing agent comprises at least one peroxide chosen from inorganic peroxyhydrated salts.

32. The smoke composition as claimed in claim 1, characterized in that said base comprises from 3 to 15% by weight of starch.

33. The smoke composition as claimed in claim 1, characterized in that it comprises from 6 to 12% by weight of silica.

34. The smoke composition as claimed in claim 1, characterized in that said base comprises from 2 to 7% by weight of a chlorate or of a perchlorate or of an iodate or of a periodate.

35. The smoke composition as claimed in claim 1, characterized in that said base comprises from 2 to 7% by weight of a nitrite.

36. The smoke composition as claimed in claim 1, characterized in that said base comprises from 35 to 45% by weight of ammonium nitrate, from 5 to 12% by weight of sodium nitrate or potassium nitrate, from 6 to 12% by weight of starch, from 4 to 9% by weight of a hydroxycarboxylic acid or of its salt containing at most 6 carbon atoms, from 7 to 10% by weight of silica, and the rest in clay.

37. The smoke composition as claimed in claim 18, characterized in that said base also comprises one or more halogenated salts, in a proportion ranging from 1 to 7% by weight of the base.

38. The smoke composition as claimed in claim 1, characterized in that the active ingredient(s) is (are) chosen from bactericidal, fungicidal or insecticidal ingredients.

39. The smoke composition as claimed in claim 1, characterized in that at least one of the active ingredients is absorbed, in the liquid or pasty state, onto at least one regulating compound.
